(19) United States
(12) Patent Application Publication
(10) Pub. No.: US 2021/0057075 A1
(43) Pub. Date: Feb. 25, 2021

(54) Market Triggered Surgical Supply Chain and Financial Management

(71) Applicant: Allevion, Inc., Aventura, FL (US)

(72) Inventors: Arnon Krongrad, North Miami, FL (US); Kimberly Susan Langer, Hallandale Beach, FL (US)

(21) Appl. No.: 16/865,698

(22) Filed: May 4, 2020

Related U.S. Application Data
(63) Continuation of application No. 15/882,192, filed on Jan. 29, 2018, now abandoned.
(60) Provisional application No. 62/456,038, filed on Feb. 7, 2017.

(51) Int. Cl. G16H 20/40; G06Q 10/08; G16H 40/20

(52) U.S. Cl. CPC G16H 20/40; G06Q 30/0641; G16H 40/20; G06Q 10/087

(57) ABSTRACT

An example method for presenting invasive and/or operative procedure bundles can include: receiving a request for an invasive and/or operative procedure bundle, the request identifying an end user and one or more services requested by the end user; determining which kind of participants are required; determining which specific participants will participate; converting requests for the specific participants into electronic data interchange (EDI) transactions; and communicating to the specific participants using the EDI transactions to define resource needs and deliver specifics.

### RELATED APPLICATION(S)

This application claims the benefit of U.S. Patent Application Ser. No. 62/456,038 filed on Feb. 7, 2017, the entirety of which is hereby incorporated by reference.

This application is related to U.S. Patent Application Ser. No. 61/872,962 filed on Sep. 3, 2013 and U.S. patent application Ser. No. 14/657,146 filed on Mar. 13, 2015, the entireties of which are hereby incorporated by reference.

### BACKGROUND

The high cost and limited availability of invasive and/or operative procedures may present barriers to their delivery to patients, be it directly or indirectly through third-party payors. High price and limited availability cause patients to delay and altogether skip the care they need.

Delivery of an invasive and/or operative procedure requires the carefully synchronized, coordinated, matched, and complete bundling of highly-specialized and specific human and material components, including providers, such as surgeons, anesthesiologists, and pathologists; environments, such as operating rooms and endoscopy suites; and equipment, such as power drills; and instruments. These components of invasive and/or operative procedures may be found in uncoordinated and different locations. This leads to inefficiencies and price increases.

Further, patients, surgeons, facilities, payers, device makers, and supply chain vendors can be poorly coordinated. For example, manufacturers of orthopedic implants often rely upon distributed networks of distributors, who in turn rely upon networks of field representatives for delivery of implants. This can result in inadequate tracking and transporting of inventory. Such manufacturers interact with surgical facilities, who request their implants, typically through these distributors and representatives, and who pay manufacturers slowly, often after only receiving payment from a third-party payer.

In this scenario, neither facility, nor manufacturer, nor distributor, nor representative is communicating with the patient, who is the true source of the demand. In this scenario, manufacturers keep much more inventory, operate much more warehouse space, and engage many more distributors and representatives than would rationally be needed. Such fragmentation—decoupled as it is from demand triggers—can drive up the cost of surgery and reduce manufacturer profitability.

### SUMMARY

Embodiments of the disclosure are directed to methods implemented on an electronic computing device for presenting invasive and/or operative procedure bundles comprising: receiving a request for an invasive and/or operative procedure bundle, the request identifying an end user and one or more services requested by the end user; determining which kind of participants are required; determining which specific participants will participate; converting requests for the specific participants into electronic data interchange (EDI) transactions; and communicating to the specific participants using the EDI transactions to define resource needs and delivery and payment specifics

The details of one or more techniques are set forth in the accompanying drawings and the description below. Other features, objects, and advantages of these techniques will be apparent from the description, drawings, and claims.

### DESCRIPTION OF THE DRAWINGS

FIG. 1 shows an example system that supports bundling medical provider services.

FIG. 2 shows example modules of the web server computer of FIG. 1.

FIG. 3 shows an example user interface that may be rendered on a client computer of FIG. 1.

FIG. 4 shows an enlarged view of the interactive map of the user interface of FIG. 3.

FIG. 5 shows enlarged views of the tables of the user interface of FIG. 3.

FIG. 6 shows another example user interface that may be rendered on a client computer of FIG. 1.

FIG. 7 shows an enlarged view of the interactive map of the user interface of FIG. 6.

FIG. 8 shows enlarged views of the tables of the user interface of FIG. 6.

FIG. 9 shows yet another example user interface that may be rendered on a client computer of FIG. 1.

FIG. 10 shows an example certificate of complications protection.

FIG. 11 shows another example certificate of complications protection.

FIG. 12 shows an example user interface for financing options.

FIG. 13 shows a flowchart for an example method implemented on the web server computer of FIG. 1 for bundling invasive and/or operative procedures.

FIG. 14 shows a flowchart for an example method for providing credentials and privileges for a provider.

FIG. 15 shows example physical components of the web server computer of FIG. 1.

FIG. 16 shows an alternative example user interface that may be rendered on the client computer of FIG. 1.

FIG. 17 shows an enlarged view of the interactive map of the user interface of FIG. 16.

FIG. 18 shows another example method for facilitating a bundled invasive and/or operative procedure.

### DETAILED DESCRIPTION

The present disclosure is directed to systems and methods for bundling medical provider services, facilities, implants and equipment in order to produce and present a plurality of new choices of service bundles and increase convenience and transparency, and reduce price to the individual or entity at financial risk for the surgery. Facing the bundled service choices produced by these systems and methods, an end user who is looking for an invasive and/or operative procedure may select from a plurality of bundled service choices the one that best meets his/her needs. Components of bundles of invasive and/or operative procedures can include: an appropriately credentialed and privileged physician to perform the procedure, a facility in which the procedure may be performed, a date for the procedure, and a price for the procedure.

Some procedures that do not create a break in the skin, such as colonoscopies, are commonly referred to as “invasive.” Other procedures, for example surgical procedures that create a break in the skin, are commonly referred to as “operative.” The procedures described herein include both invasive and/or operative types such as knee replacements, hip replacements, hysterectomies, sleeve gastrectomy, cardiac catheterization, and colonoscopies; other procedures are possible.

The providers that perform the procedures may include surgeons, surgical assistants, physician assistants, anesthesiologists, nurse anesthetists, gastroenterologists, pathologists, radiology technicians, etc. Such providers are credentialed and privileged at the facility at which the procedure is to be performed; primary documents, such as medical licenses, board certifications, etc. are verified.

The facilities at which invasive and/or operative procedures may be performed include hospitals, ambulatory surgery centers, endoscopy centers, radiology centers, medical centers and physician offices. The facilities are generally located in a well-defined geographical area, such as a state. However, the facilities may be in various locations within the geographical area. Equipment used for the procedure may be resident at the facility or may be transported to the facility from other locations for the operative episode.

The bundled services are presented to the end user via a computer interface, typically via a web page or data import into a client legacy environment that may be accessed by the end user. As discussed in more detail later herein, the web page provides a map showing the specific location of the bundled service, provides detailed information regarding the bundles and permits a selection of a bundle by the potential patient.

The systems and methods also provide for protection against economic losses due to complications that may arise during and/or after the invasive and/or operative procedure, to address the psychological barrier to care—fear of complications—and to give the patient peace of mind (“complications protection”). In this example, protection against complication-related financial losses is automatically included in those procedures for which it is available. In other examples, the protection is provided as an option. In addition, the systems and methods also provide access to financing options for the potential patient to minimize financial barriers to care.

The systems and methods are directed to three types of clients: uninsured patients, insured patients, self-insured employers (i.e., Carnival Cruise Lines) and other third party payors. One example of a third party payor is an insurer for the patients. An example of an insurer is Blue Cross Blue Shield of Florida, also known as Florida Blue.

The systems and methods make use of a computer algorithm to compile information regarding available providers, facilities, implants and equipment and to create bundled services of invasive and/or operative procedures that are made available to the end user via the web site or data import. The web site is searchable, for example permitting an uninsured end user to search for availability for a specific procedure at a specific location at a specific price and time. The computer algorithm provides for sorting and filtering of various combinations of providers, locations and price.

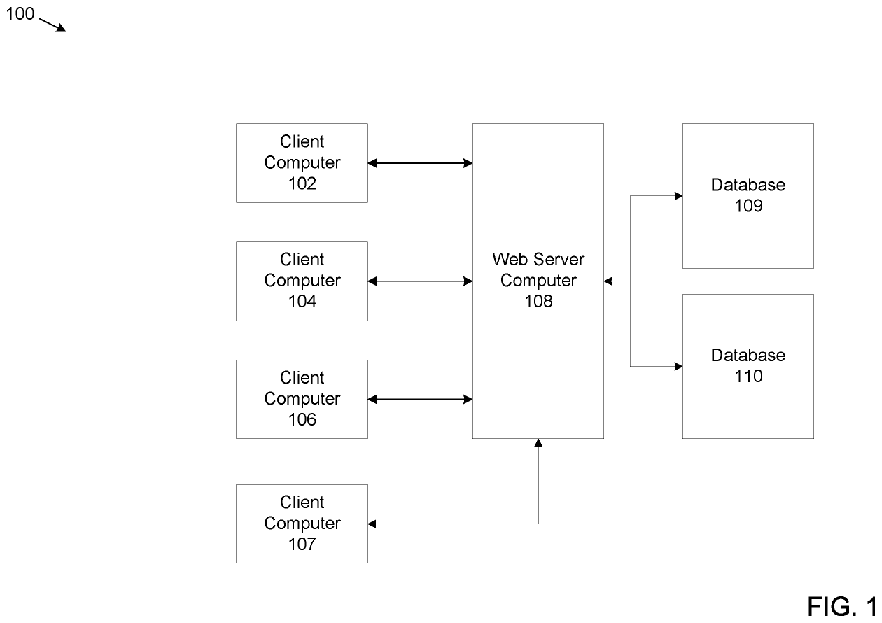

FIG. 1 shows an example system 100 that supports bundling of invasive and/or operative procedure bundles. System 100 includes client computers 102, 104, 106, 107, web server computer 108 and database 110.

Client computers 102, 104, 106 and 107 may be desktop, laptop or tablet computers, smart telephones, and/or other interactive digital products. Client computers 102, 104, 106 and 107 permit a user to access a web site that includes information on the invasive and/or operative procedure bundles. For the example system 100, client computer 102 is used by an uninsured patient, client computer 104 is used by an insured patient, client computer 106 is used by a payor, for example by an employee of Florida Blue, and client computer 107 is used by a member of a third party payor plan via the payor's user portal (as described below). More or fewer client computers may be used.

Web server computer 108 is a server computer that hosts the web site that may be accessed via client computers 102, 104 and 106 and 107. Database 109 is a central repository used to store patient information submitted via the online consultation request form. The web site provides information regarding the invasive and/or operative procedure bundles that may be rendered on a web browser of client computers 102, 104, 106 and 107. Database 110 also includes one or more computer algorithms that are used to compile the invasive and/or operative procedure bundles from information that may be pushed to and stored on web server computer 108, on database 110 or on other server computers or databases.

Database 110 is a database that includes information on providers, facilities, vendors and equipment, as well as pricing for the same. Providers include available surgeons, assistants, anesthesiologists and other physicians. Facilities include available hospitals, medical centers and physician offices. Implants include any necessary components needed to perform a specific invasive and/or operative procedure. Equipment includes any equipment needed to perform a specific invasive and/or operative procedure. Some or all of the equipment may be located at one of the facilities. Some or all of the equipment may be located elsewhere and may need to be transported to one of the facilities. Algorithms referenced in 0034 are used to identify and fulfill equipment needs for a specific invasive and/or operative procedure at a specific facility.

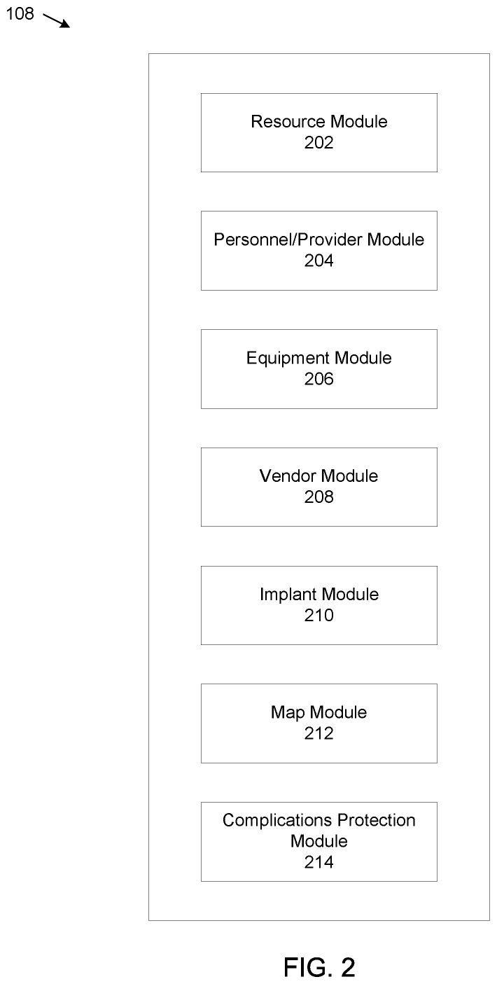

FIG. 2 shows example module of web server computer 108. The example modules include a resource module 202, a personnel module 204, an equipment module 206, a vendor module 208, an implant module 210, a map module 212 and a complications protection module 214.

Other modules are possible. For example, another module can be programmed to track dates and provide pricing for different invasive and/or operative procedure bundles based upon the particular date and time. In yet other examples, such a module can assist in scheduling of the different aspects of the invasive and/or operative procedure bundles, including patient visits and/or location/surgical components.

The example resource module 202 is programmed to identify medical facilities including qualified, credentialed, and privileged providers, operating room capabilities, associated procedure fees and facility assets that may be used as part of the invasive and/or operative procedure bundles. The medical facilities and associated characteristics thereof are obtained from database 110. The resource module 202 can also

The example personnel module 204 is programmed to identify medical providers that may be used as part of the invasive and/or operative procedure bundles. As part of the identification process, the personnel module 204 also verifies credentials for the identified medical providers, determines facilities where the medical providers have privileges and determines availability for the medical providers. Personnel may perform more than operative or invasive procedure at more than one facility. Personnel module 204 also includes the fee for the specific provider at a specific location for a specific procedure. Personnel module 204 also includes information such as personal statements, administrative contacts. More elements may be added.

In some examples, the personnel module 204 also captures other metrics about the medical providers. For example, in the context of a physician, the personnel module 204 can be programmed to capture outcome-related quality metrics over time on a per-location or per-physician basis. One example of such a metric is a clinical outcome like intraoperative blood loss. Other objective and/or subjective metrics can be tracked and provided to the end user when selecting among bundles.

The example equipment module 206 is programmed to identify equipment that is needed to perform the invasive and/or operative procedures. The equipment module 206 also determines location and availability of the identified medical equipment, specifically determining whether the identified equipment is located and available at the facilities identified by the resource module 202.

The example vendor module 208 is programmed to identify equipment and implant vendors that may be used to obtain medical equipment and implantable devices that may not be available at the facilities identified by the resource module 202. The identified equipment vendors are also vendors that have the capability of transporting the medical equipment to facilities where the medical equipment is needed.

The example implant module 210 is programmed to identify availability of implants that may be needed during the invasive and/or operative procedures. Examples of implants that may be needed include artificial knee replacements and artificial hip replacements. Other implants may be used. It is also noted that surgeons have preferences regarding the brand/type of implant. This characteristic is factored into creation of the bundle.

More specifically, the implant module 210 can be configured to determine what implant is needed and configure procurement and/or delivery of that implant to the desired participant and/or location. For example, the system determines which participants are necessary. The implant module 210 is programmed to then determine which implant or implants are needed and how to most efficiently locate and deliver those implants.

This can include the implant module 210 being configured to communicate with one or more logistics providers (e.g., supply chain vendor(s)) to accomplish the identification and delivery of the implants. For example, the implant module 210 is programmed to communicate with a logistics provider, like FedEx Corp., DHL Logistics, UPS Supply Chain Solutions, Amazon, Uber, Otto etc., using a common messaging scheme, like the Electronic Data Interchange (EDI) communication method.

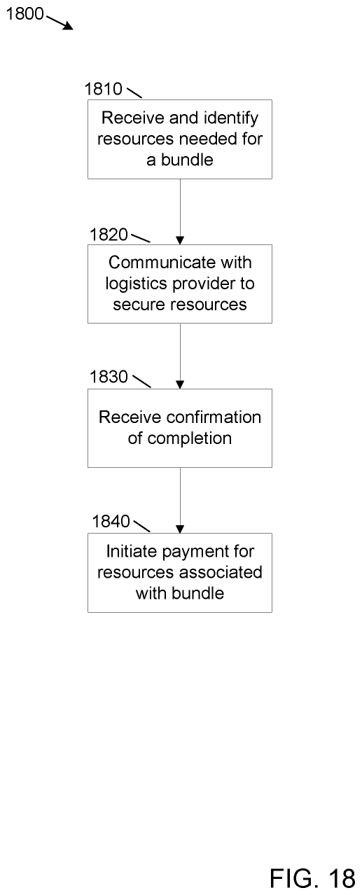

Through various EDI communications between the implant module 210 and the logistics provider (see FIG. 18 below for more details), the implant module 210 communicates to the specific participants with where and when the medical device(s), such as a knee implant, need to be delivered by the logistics provider.

In this embodiment, the resource module 202, personnel module 204, equipment module 206, implant module 210 and vendor module 208 are used to create the price for the bundle of medical services. In this example, the price is the actual cost to the patient for the medical service (including any fees associated with the use of the system 100. For example, each facility may charge a different amount for use of a particular asset, such as an operating room. Further, each medical provider can charge different amounts based upon, for example, the type of procedure and location at which the procedure will occur.

Further, in this embodiment, the credentialing and privileging of the medical provider at each of the facility locations is tracked. In one example, there is an automated process that allows the provider to obtain privileges at multiple facilities. This allows for greater flexibility and efficiency in the matching of the locations at which a provider can deliver services resulting in new services previously unavailable to the end user.

The example map module 212 is programmed to render an interactive map on a user interface of client computers 102, 104, 106 and 107. The interactive map shows locations where invasive and/or operative procedure bundles are available and provides related details.

The example complications protection module 214 is programmed to compile complications protection that may be presented to patients. The complications protection provides varying levels of coverage to provide patients with protection from the financial losses associated with complications arising during and after the procedures.

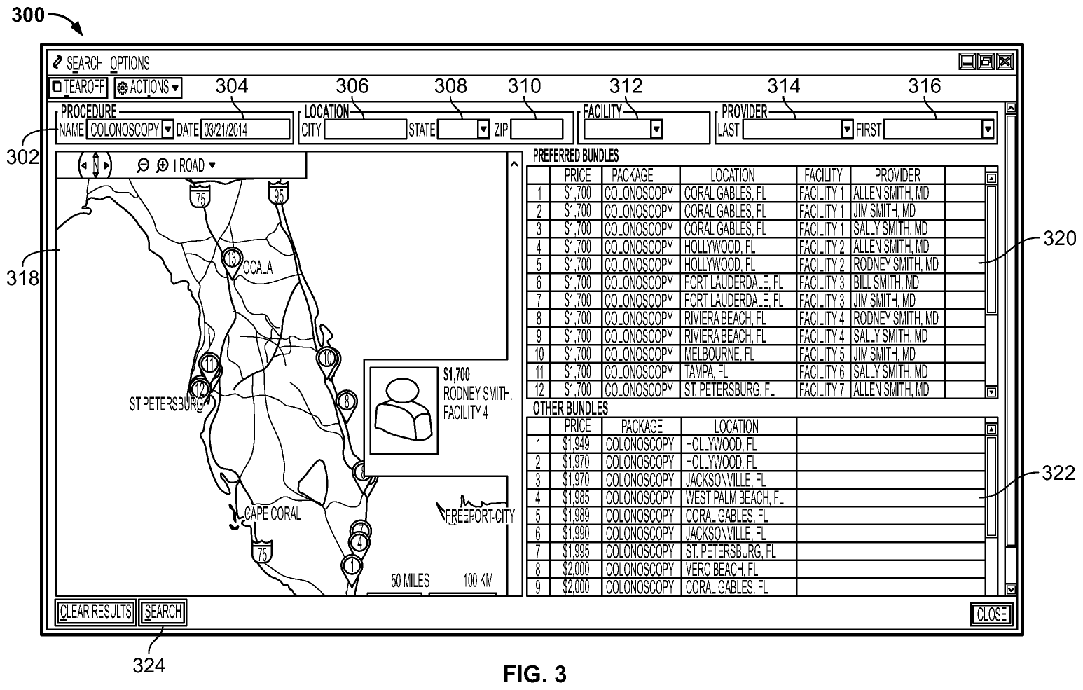

FIG. 3 shows an example user interface 300 that may be rendered for the end user on a web browser of client computers 106. The end user may be an uninsured patient or a third party payor, such as an insurance company. The example user interface 300 shows a result of a search for particular procedure bundles (e.g., a colonoscopy) in a specific geography (e.g., the state of Florida).

The user interface 300 includes search fields for the service bundle including search fields for a procedure 302, a date 304, a location including a city 306, a state 308 and a zip code 310, a facility 312 and a provider including a last name 314 and first name 316. The user interface 300 also includes an interactive map 318, a table 320 of service bundles from preferred bundles, a table 322 of service bundles from other bundles, and a search button 324.

The user interface 300 shows a result of search for bundled invasive and/or operative procedures on a specified date (e.g., Mar. 21, 2014. The end user at client computer 102 or 106 selects the procedure from procedure field 302. A drop down list box (not shown in FIG. 3) permits the end user to select the procedure from a list of available procedures. The date field 304 permits the payor to enter a specific date for the procedure. The payor then selects search button 324. The payor can also search for and then possibly filter and sort a specific location, facility and provider.

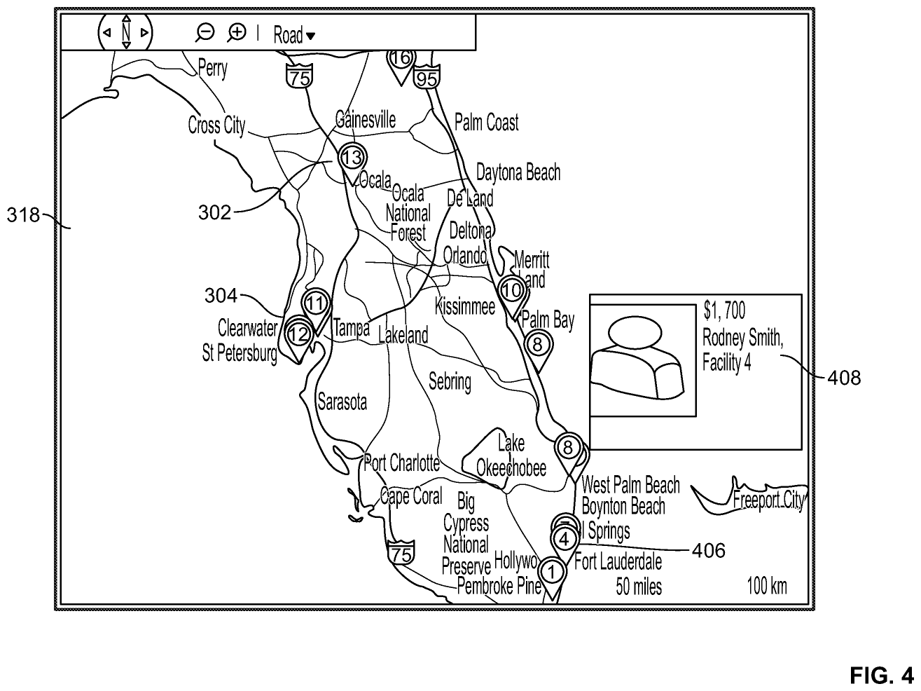

FIG. 4 shows an enlarged view of interactive map 318. Interactive map 318 is displayed as result of a search for available procedure bundles for any given date in the state of Florida. As shown in FIG. 4, each available bundle is indicated by a numbered marker. For example marker 402 shows a marker numbered 13, indicated that a colonoscopy bundle is available on Mar. 21, 2014 in Ocala, Fla. Similarly, marker 404 shows a marker numbered 12, indicating an available colonoscopy bundle in St. Petersburg, Fla. and marker 406 shows a marker numbered 4, indicating an available colonoscopy bundle in Riviera Beach, Fla. In these examples, the example marker numbers 13, 12 and 4 correspond to identifiers for specific medical facilities.

When any one of these markers is selected, a pop-up window is displayed providing more information about the bundle at the marker location. For example, when marker 406 is selected, an example pop-up window 408 is displayed. Pop-up window 408 shows that a colonoscopy bundle at Riviera Beach Fla. is to be performed at facility 4 by Dr. Rodney Smith. The price of the procedure, including complications protection is $1,700. Other information, such as details about the service provider (e.g., details about the surgeon and facility) may also be provided.

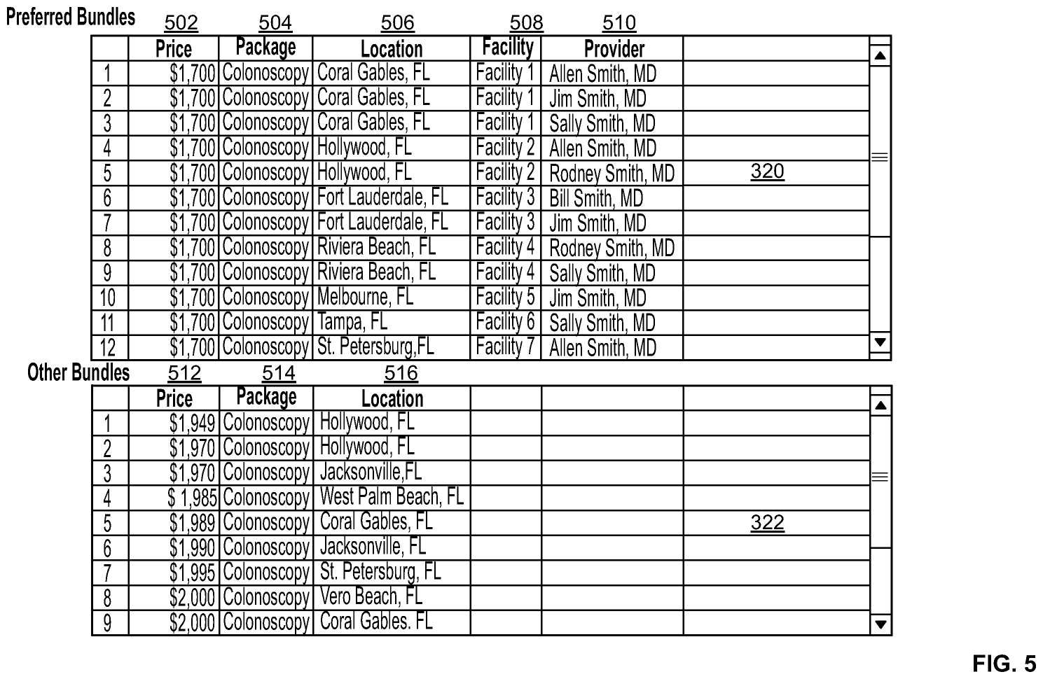

FIG. 5 shows enlarged views of example tables 320 and 322. Table 320 shows price comparisons for different service bundles for a preferred provider. Each bundle consists of a price (502) for a specific procedure (504), in this case a colonoscopy, at a specific location (506), at a specific facility (508) with a specific provider (510). The provider is a physician performing the procedure. Table 320 includes 12 procedure bundles. As shown, bundles are available at seven different cities within the state of Florida and at seven different facilities, each facility corresponding to a specific city.

In some examples, the tables 320, 322 are presented to end users or other users of the system (see “white-label” implementation described below) below. In other examples, the tables 320, 322 are not presented to end users in the format shown.

Each service bundle within a specific city includes a different provider. However, certain providers may perform procedures at multiple locations. For example, service bundles are provided for Allen Smith at facility 1 in Coral Gables, at facility 2 in Hollywood and at facility 7 at St. Petersburg.

Table 322 provides price comparisons for the same colonoscopy procedure bundle but for other providers. Table 322 includes nine different options including the price (512), specific procedure (514) and the location (516). As shown in table 322, prices for the other provider bundles range from $1,949 to $2,000, each of which is higher than the $1,700 bundle price.

In one example, the tables 320, 322 represent the bundles of medical services associated with different health plans. In other examples, the tables can be used to present prices associated with bundles that fall outside of medical coverage of a patient, along with bundles associated with the patient's medical insurance coverage.

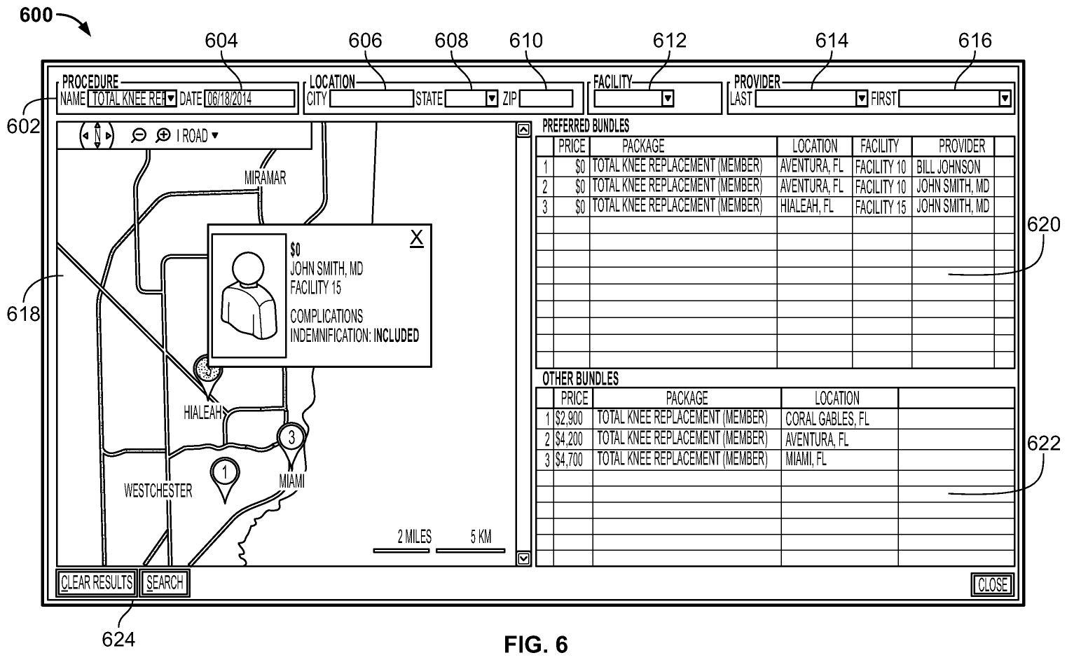

FIG. 6 shows an example user interface 600 for an insured patient that may be rendered on a web browser of client computer 104. The example user interface 600 shows a result of a search for a total knee replacement bundle in the state of Florida with out of pocket amounts for an insured patient rendered using EDI benefit determination transactions.

With user interface 600, a search can be made for a specific procedure (602), a specific date (604) a specific location including a city (606), a state (608) and a zip code (610, a specific facility (612) and a specific provider including a last name (614) and a first name (616) and other provider-specific details. User interface 600 also includes an interactive map 618, a table 620 of service bundles from preferred bundles, a table 622 of service bundles from other bundles and a search button 624.

The user interface 600 shows a result of search for bundled total knee replacement on Mar. 21, 2014. The insured patient at client computer 104 selects the total knee replacement procedure from procedure field 602. A drop down list box (not shown in FIG. 6) permits the insured user to select the total knee replacement procedure from a list of available procedures. The date field 604 permits the insured user to enter a specific date for the procedure. Bundle pricing may fluctuate depending upon the date of the query. Price date sensitivity is accommodated in the bundle generation. The insured user then selects search button 324. The insured user can also search for a specific location, facility and provider.

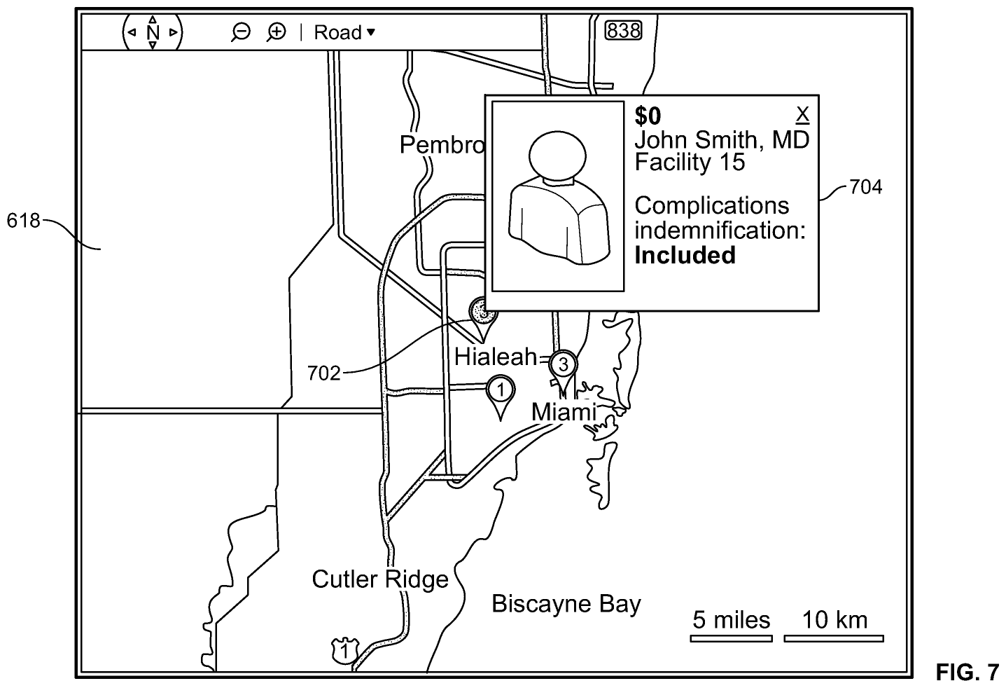

FIG. 7 shows an enlarged view of interactive map 618. Interactive map 618 is displayed as result of a search for available total knee procedure bundles on Mar. 21, 2014 in the state of Florida. As shown in FIG. 7, each available bundle is indicated by a numbered marker. For example marker 702 shows a marker numbered 3, indicated that a total knee replacement bundle is available on Mar. 21, 2014 in Hialeah, Fla.

When marker 702 is selected, a pop-up window is displayed providing more information about the bundle at the marker 702 location. For example, when marker 702 is selected, an example pop-up window 704 is displayed. Pop-up window 704 shows that a total knee replacement procedure bundle at Hialeah Fla. is to be performed at facility A by Dr. John Smith. The pop-up window 704 also shows that the price of the procedure includes complications protection.

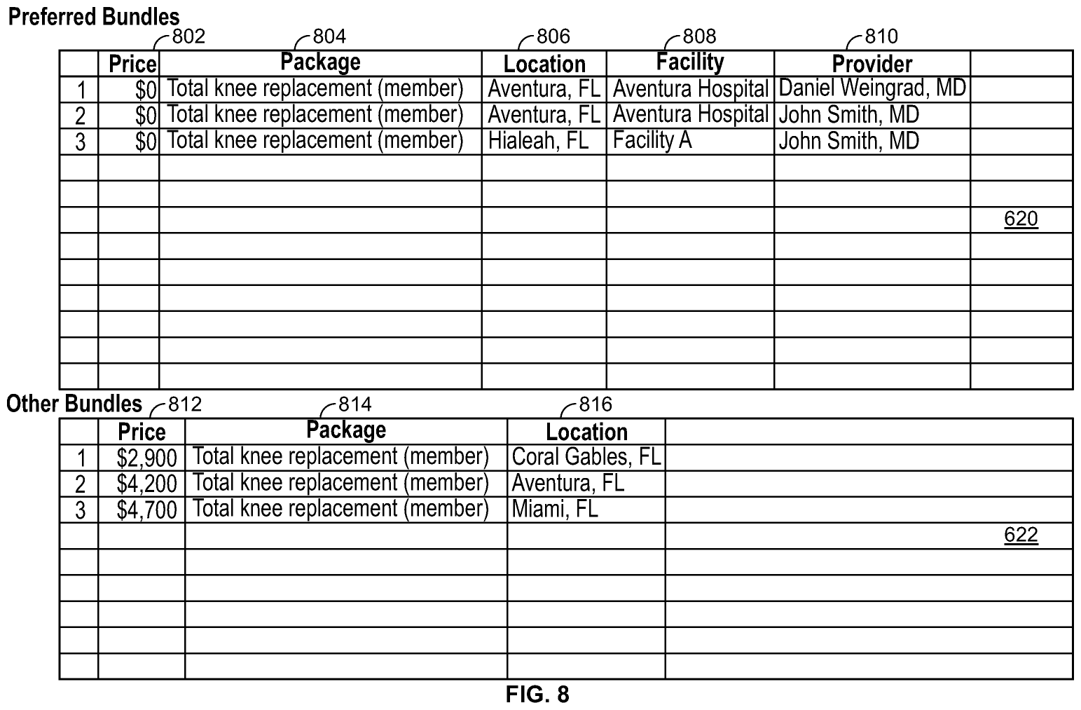

FIG. 8 shows enlarged views of example tables 620 and 622. Table 620 shows price comparisons for different service bundles for a preferred provider. Each bundle consists of a price (802) for a specific procedure (804), in this case a total knee replacement, at a specific location (806), at a specific facility (808) with a specific provider (810). The provider is a physician performing the procedure.

Table 620 includes three bundles. Because table 620 is for an insured patient, the price 802 represents an out-of-pocket cost for the patient based upon that patient's insurance benefits. For example, the patient may have no out-of-pocket costs, such as a deductible or cost share, for a particular invasive and/or operative procedure. As shown in FIG. 8, under this kind of benefit design, for preferred bundles the associated out-of-pocket cost for each is zero, whereas for other bundles the associated out-of-pocket cost for each is other than zero. These out-of-pocket costs are automatically calculated based upon patient-specific financial data kept by and procured from the insurance company using standardized Electronic Data Interchange (EDI) transactions and provided in the patient-specific tables 620, 622.

As shown, service bundles are provided at two different cities within the state of Florida and at two different facilities, each facility corresponding to a specific city. Each service bundle within a specific city includes a different provider. However, certain providers may perform procedures at multiple locations. For example, service bundles are provided for Dr. John Smith at Aventura Hospital in Aventura, Fla. and at Facility A at Hialeah, Fla.

Table 622 provides price comparisons for total knee replacement service bundles. Table 622 includes three different options including the price (812), specific procedure (814) and the location (816). As shown in table 622, prices for the other bundles range from $2,900 to $4,700.

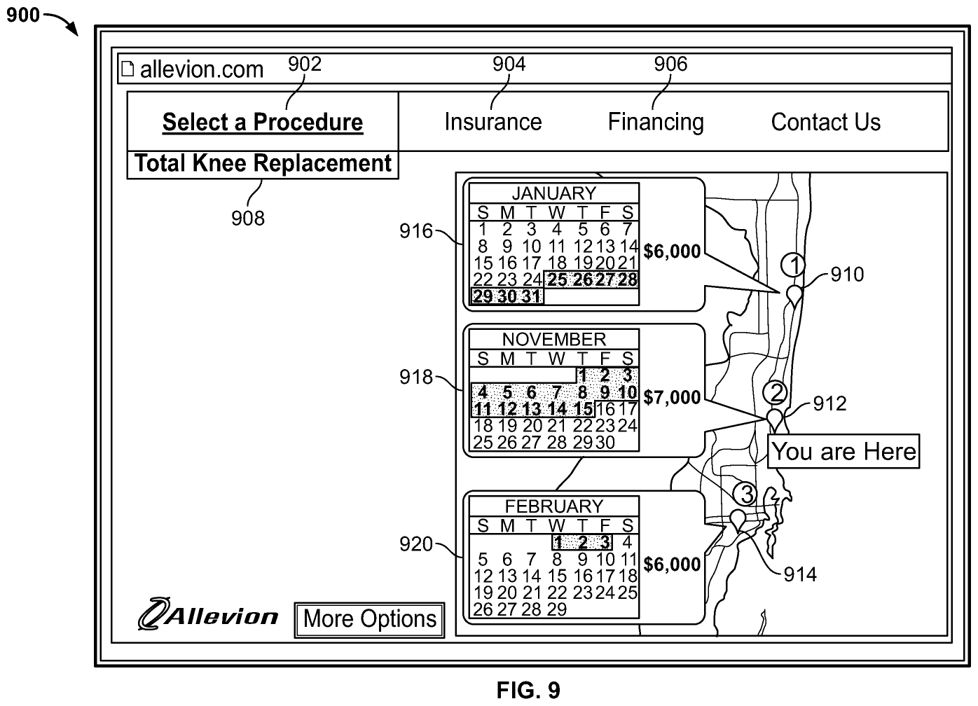

FIG. 9 shows an example alternative user interface 900 for an uninsured patient that may be rendered on a web browser of client computer 102. The alternative user interface 900 may be used in some embodiments. The example user interface 900 shows a result of a search for total knee replacement bundles in the state of Florida.

The example user interface 900 includes link 902 for selecting a procedure, link 904 for selecting complications protection and a link 906 for selecting financing options. When procedure 908 for a total knee replacement is selected, the user interface display shown in FIG. 8 is rendered on the web browser of client computer 102.

As shown in FIG. 9, an interactive map is displayed showing options for the uninsured patient. The interactive map displays markers 910, 912 and 914. Each marker corresponds to a city in which the total knee replacement bundle is available and the price with which it is associated. For example, marker 910 indicates Boynton Beach, Fla., marker 912 indicates Fort Lauderdale, Fla. and marker 914 indicates Coral Gables, Fla.

In the embodiment shown in FIG. 9, an information area is displayed for each of markers 910, 912 and 914. Information area 916 is associated with marker 910, information area 918 is associated with marker 912 and information area 920 is associated with marker 914. Each information area includes a calendar showing highlighted days on which the procedure may be performed and a price for the procedure. The uninsured patient can click a date on one of the information areas and sign up for the bundle on the date and location selected.

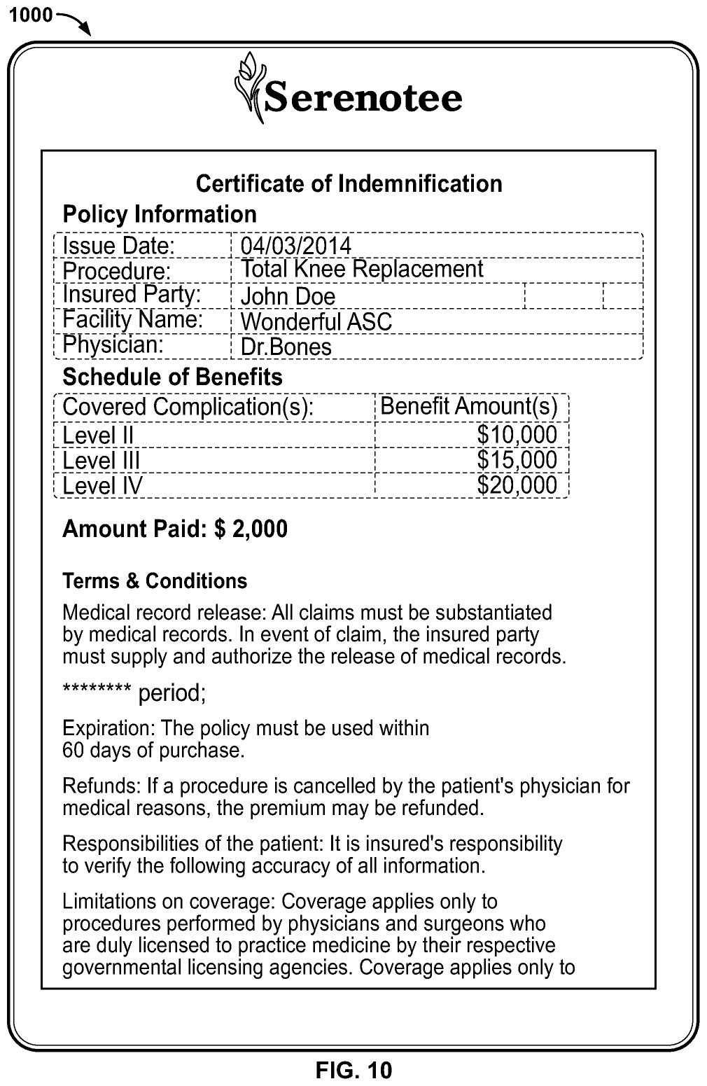

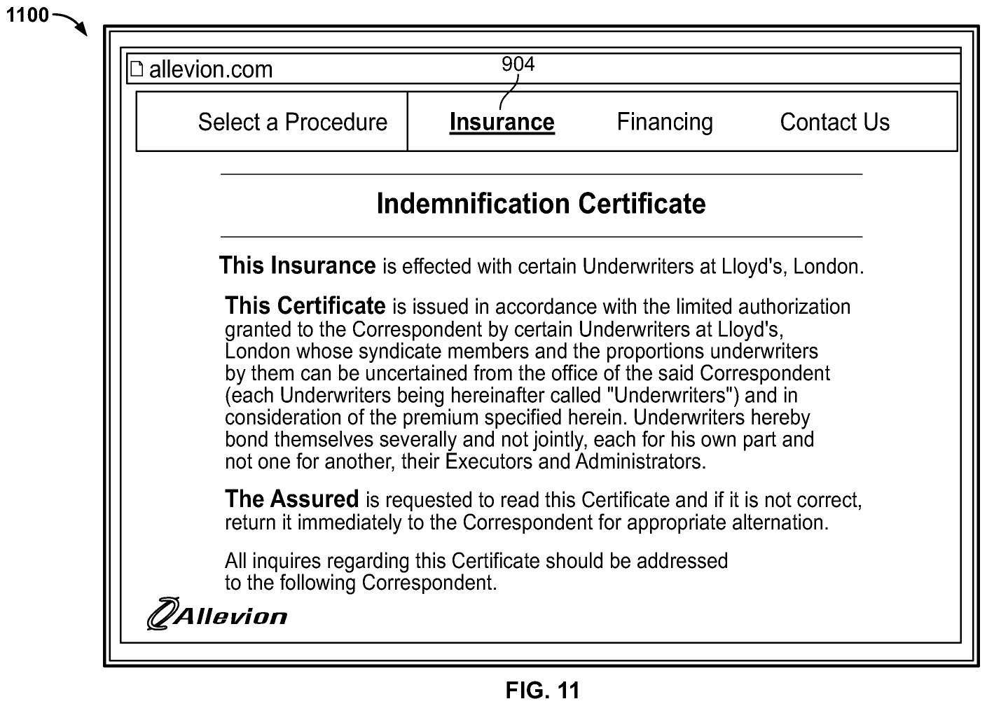

User interface 900 also permits the uninsured user to sign up for complications protection. The complications protection provides financial protection against complications associated with the procedure. FIG. 10 shows an example certificate of complications protection 1000. The example certificate of complications protection 1100 shows that the user paid $2,000 for the coverage. Also shown are policy information, a schedule of benefits and terms and conditions of the coverage. An example certificate of complications protection is shown in FIG. 11.

In examples provided herein, the prices of complications protection are developed using a pre-operative assessments of risks of pre-operative and intra-operative accidents and/or complications adversely affecting the health and/or functional status of patients going for operative and/or invasive procedures to pre-operatively price and qualify patients for insurance against possible accidents and/or complications.

In one example, this assessment involves two steps. The first step, risk assessment, identifies the types and severities of accidents and complications that are possible for a given operative or invasive procedure and produces an estimation of their likelihood. The risk assessment is determined upon analysis of relevant human and physical factors relating to the patient, medical personnel, e.g. surgeon and anesthesiologist, type of procedure, facility, and more.

The second step, pricing and qualification for insurance against accidents and/or complications of operative and/or invasive procedures, can apply to the output of the first step and other similar real-world scenarios and assessments, wherein a financial model and system is produced. The financial model, which can rely on the first step, forms risk pooling from individual patients about to have operative and/or invasive procedures. The newly formed risk pool and associated individual policy premiums provide the funds for payments to patients who have suffered accidents and/or complications and have made claims on that basis.

In the example shown, the complications protection is provided according to a set premium price that is added to the total price of the clinical service bundle that is presented to the patient. In other examples, the complications protection is provided as an option for the patient to select as desired.

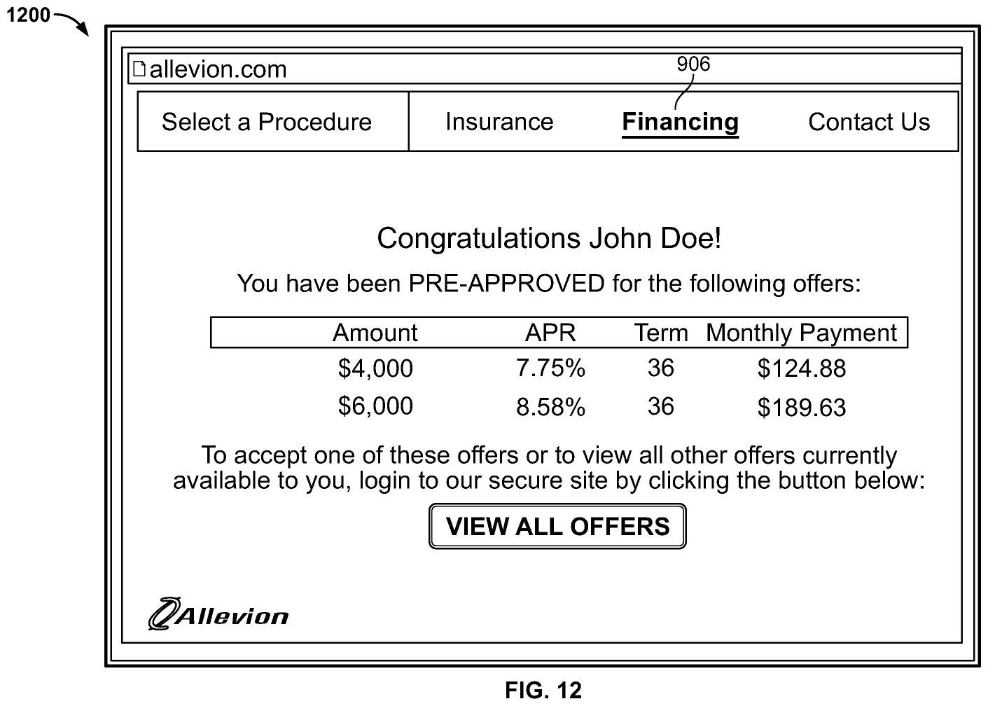

User interface 800 also permits the uninsured user to obtain financing for the selected procedure. FIG. 12 shows an example web page 1200 showing financing offers that have been preapproved for a patient. Web page 1200 shows that two offers have been approved for the patient—one for $4,000 and another for $6,000. Other information is shown for the offers including APR, term of offer and monthly payment.

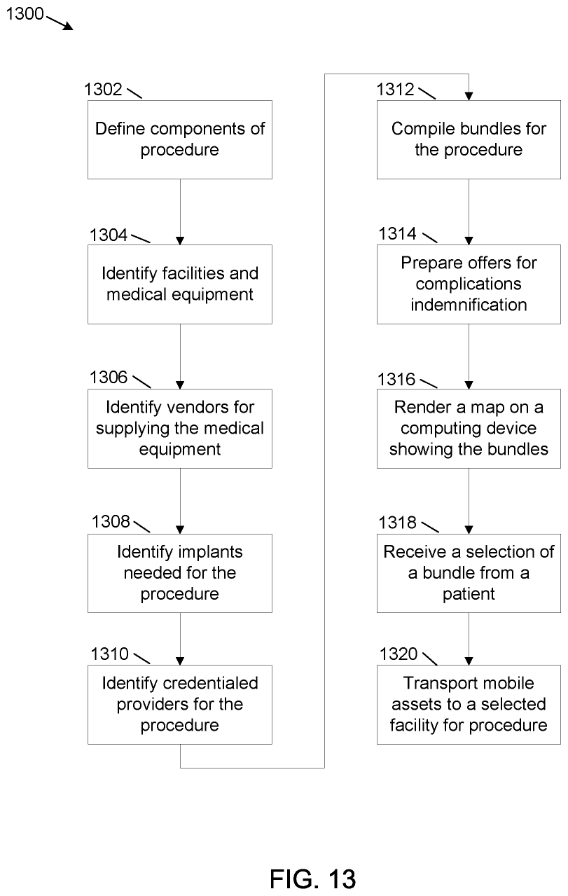

FIG. 13 shows a flowchart of an example method for bundling the components of invasive and/or operative procedures. Examples of procedures that may be effectively composed by bundling their components include total knee replacements, hip replacements, hysterectomies, gastric sleeves and colonoscopies.

At operation 1302, the required components of an invasive and/or operative procedure are defined. These can include, for example, the surgeon, facility, anesthesia, implant, etc.

At operation 1304, the available facilities for invasive and/or operative procedures are identified. The facilities are typically hospitals, ambulatory surgery centers, endoscopy centers, radiology centers, cardiac catheterization laboratories, or provider offices. The identified facilities are ones that have excess capacity and that may be used for bundled invasive and/or operative procedures. The facilities are identified for specific times and dates at which they are available. The facilities may be located in different cities, but are typically located in a defined geographical area such as a state.

In addition, equipment needed for the invasive and/or operative procedures is identified. Some of the equipment may be available at the facilities identified at operation 1302. Other equipment may not be available at the facilities identified at operation 1302 and may need to be transported to the facilities identified from other locations.

As part of this operation, a determination is made regarding whether or not a facility already has certain components or whether the price of the bundle will need to include the cost of providing the component for the procedure. For example, some facilities may have certain medical devices already available for use in an operating room. Others may not and would require that the medical devices be relocated to that operating room for the procedure.

At each of these steps, the prices associated with the each component are also evaluated to determine the overall price of the associated bundle. This pricing component, for example, can be used to evaluate the bundle against a price already offered by the medical facility to determine if prices or additive to the overall price.

At operation 1306, vendors are identified for supplying the identified equipment that may not be available at the identified facilities. The vendors selected are ones capable of transporting needed medical equipment to facilities selected for the invasive and/or operative procedure.

At operation 1308, implants needed for the invasive and/or operative procedures are identified. Example implants include artificial knees and penile prostheses.

At operation 1310, qualified providers, such as surgeons, are identified for the invasive and/or operative procedure. Those identified are credentialed and privileged by one or more of the identified facilities. To perform the invasive and/or operative procedure, one or more of the qualified providers can be moved from one facility in one location to another facility at different location. These providers can collectively form a mobile preferred provider organization.

A facility agnostic administrative service enables and accelerates provider mobilization to new facilities; it does this by reducing the time needed to pass new providers through facility medical staff offices, credentialing committees, medical executive committees, board level privileging sub-committees, and other bureaucratic bodies. It does this by collecting commonly-required credentialing and privileging information, such as provider name and address; collecting and verifying primary documents, such as medical school diplomas; and presenting these materials in an organized fashion to identified facility. This service supports facility based credentialing and privileging processes or accepts authority delegated to it for credentialing and privileging by the facility.

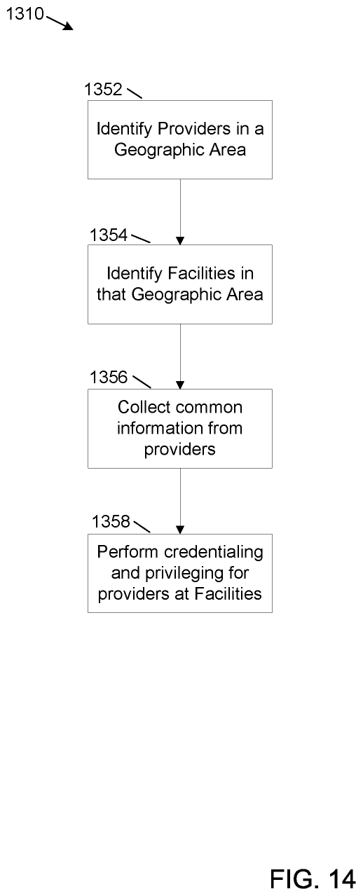

For example, as shown in FIG. 14, in one embodiment, the facility agnostic administrative credentialing and privileging service is presented to and engaged by providers as a virtual clearinghouse, through an online, interactive portal that can accept information, attestations, copies of documents, photographs, and related material for processing by the virtual medical office staff and/or transmission to a new facility for processing. The virtual medical staff office is configured to accelerate the placement of providers in different facilities in different locations. This can include, for example, obtaining credentials and/or privileges for multiple providers at multiple facilities.

Specifically, at operation 1352, the providers in a geographic area are identified. Next, at operation 1354, the facilities in that geographic area are identified. At operation 1356, information is collected from the providers that is needed for the credentialing and privileging. This can be accomplished, as noted, using an online portal or other efficient mechanism. For example, the information can be collected when a provider signs up to participate in the credentialing and privileging program. Finally, at operation 1358, the credentialing and privileging is performed in a batch style at the facilities to streamline the process.

Such a method allows for creation of the bundled and privileged provider along with the medical facility and associated price for an invasive and/or operative procedure on a computing device. The method also allows the provider to be mobile, i.e., travel to new and/or different medical facilities at different locations.

Referring again to FIG. 13, at operation 1312, components of invasive and/or operative procedures are synchronized, coordinated, and matched so as to form complete service bundles. The invasive and/or operative procedure bundles include combinations of facilities, providers, equipment, implants and available dates for the invasive and/or operative procedures. A price is also assigned to each bundle. For an uninsured patient or a payor, such as an insurance company, this is a price that is to be paid to the organization that provides the bundle. For an insured patient, the price is an out-of-pocket cost, based on the terms of his/her insurance policy.

At operation 1314, offers are prepared for complications protection for each of the service bundles. The complications protection protects patients against the economic losses that are associated with complications that may occur during or after the invasive and/or operative procedure.

At operation 1316, an interactive map is rendered on a computing device showing the bundles. Markers are sometimes used to identify locations on the map in which the bundles are available. A user can research a bundle by clicking on a marker to reveal such details as associated price and involved providers. In other embodiments, selected features are displayed directly on the map. For example, the marker can be associated with and/or replaced by the price figure in a way that does not require any more clicking to show the map and money.

At operation 1318, a selection of a bundle is received from a patient. When a user selects an invasive and/or operative procedure bundle and signs up for it, the user may be asked to fill out additional forms, for example health history forms, etc. and may also be directed to set up an appointment to visit the provider that is to perform the procedure. Information solicited will vary based upon the procedure selected. For example, a knee replacement will require different specifics of a patient's medical history than a bariatric procedure. The forms that appear upon selection of a procedure will populate based upon the selection. In addition, surgeons have varying preferences regarding medical records needed to properly “remote-qualify” a patient. For example, with total knee replacement, Physician A may prefer a standing, weight-bearing knee x-ray whereas Physician B prefers an MM.

At operation 1320, when the bundle is selected, along with arrangements to transport the providers, arrangements are made to transport mobile assets, such as medical equipment and implants, to the bundled facility so that the mobile assets arrive at the selected facility in time for the start of the procedure. In example embodiments, the system can automate one or more of these tasks. For example, the system can be programmed to automatically schedule patient visits and schedule logistics associated with the physician, location, and/or surgical components.

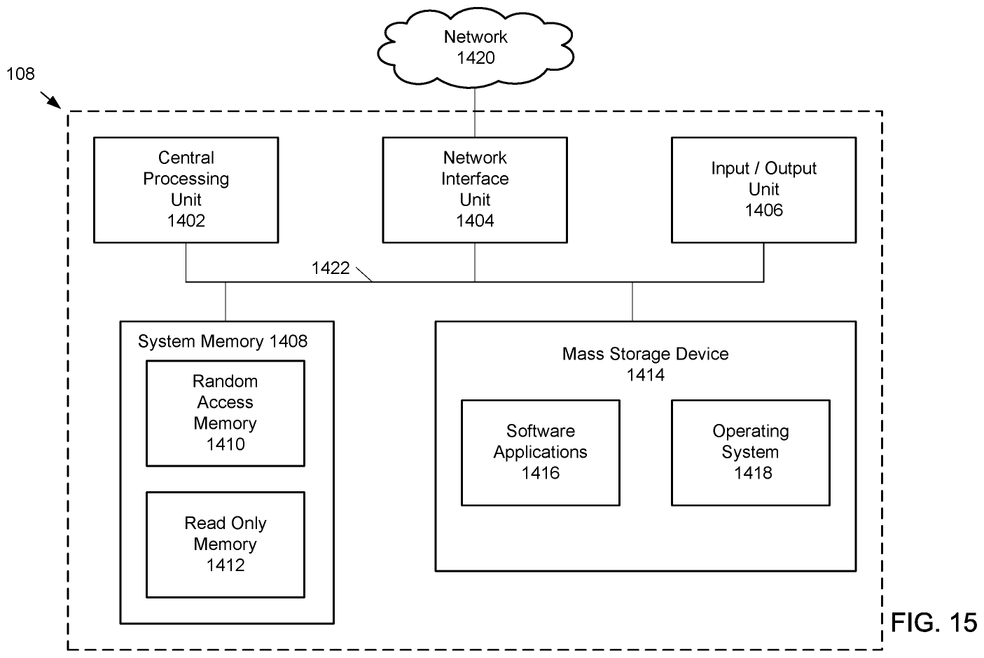

As illustrated in the example of FIG. 15, web server computer 108 includes at least one central processing unit (“CPU”) 1402, a system memory 1408, and a system bus 1422 that couples the system memory 1408 to the CPU 1402. The system memory 1408 includes a random access memory (“RAM”) 1410 and a read-only memory (“ROM”) 1412. A basic input/output system that contains the basic routines that help to transfer information between elements within the web server computer 108, such as during startup, is stored in the ROM 1412. The web server computer 108 further includes a mass storage device 1414. The mass storage device 1414 is able to store software instructions and data. A central processing unit, system memory and mass storage device similar to that in FIG. 15 are also included in client computers 102, 104 and 106.

The mass storage device 1414 is connected to the CPU 1402 through a mass storage controller (not shown) connected to the system bus 1422. The mass storage device 1414 and its associated computer-readable data storage media provide non-volatile, non-transitory storage for the web server computer 108. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the web server computer 108.

According to various embodiments of the invention, the web server computer 108 may operate in a networked environment using logical connections to remote network devices through the network 1420, such as a wireless network, the Internet, or another type of network. The web server computer 108 may connect to the network 1420 through a network interface unit 1404 connected to the system bus 1422. It should be appreciated that the network interface unit 1404 may also be utilized to connect to other types of networks and remote computing systems. The web server computer 108 also includes an input/output controller 1406 for receiving and processing input from a number of other devices, including a touch user interface display screen, or another type of input device. Similarly, the input/output controller 1406 may provide output to a touch user interface display screen or other type of output device.

As mentioned briefly above, the mass storage device 1414 and the RAM 1410 of the web server computer 108 can store software instructions and data. The software instructions include an operating system 1418 suitable for controlling the operation of the web server computer 108. The mass storage device 1414 and/or the RAM 1410 also store software instructions, that when executed by the CPU 1402, cause the web server computer 108 to provide the functionality of the web server computer 108 discussed in this document. For example, the mass storage device 1414 and/or the RAM 1410 can store software instructions that, when executed by the CPU 1402, cause the web server computer 108 to display received financial data on the display screen of the web server computer 108.

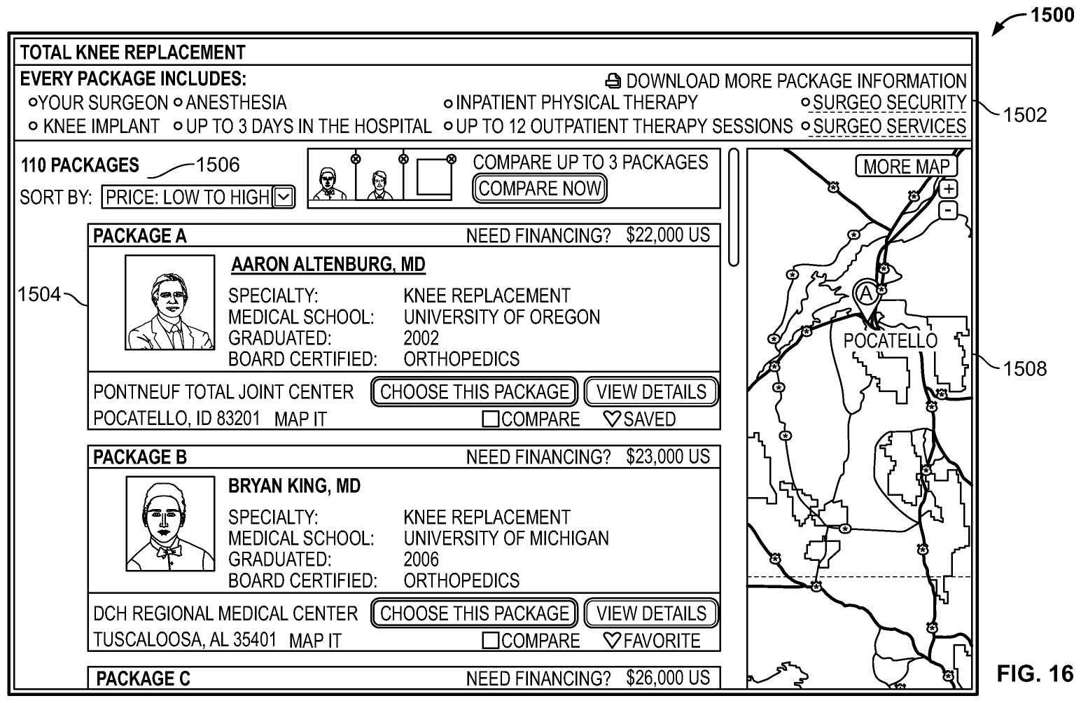

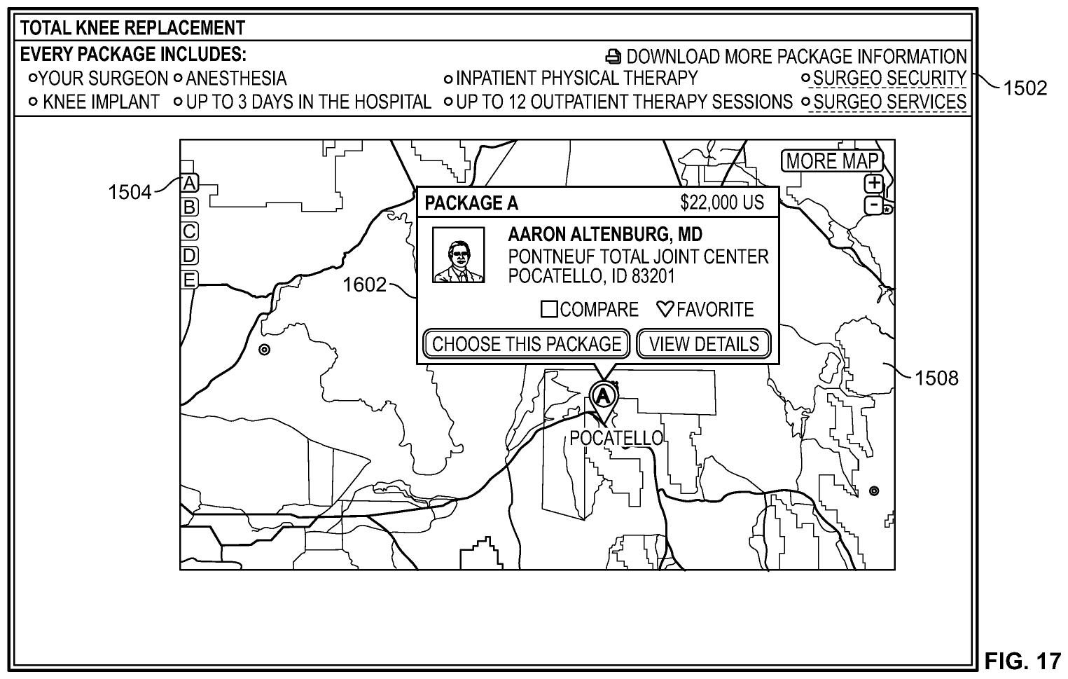

Referring now to FIGS. 16-17, another example of a user interface 1500 is shown that allows an end user to search for bundled invasive and/or operative procedures. The user interface 1500 is similar to that of the interface 300 described above, in that the interface 1500 provides a visual representation of one or more bundled invasive and/or operative procedures within a geographic region.

In this example of FIG. 16, the interface 1500 includes a detailed description 1502 of the aspects associated with the selected bundled invasive and/or operative procedure, along with a list 1504 of the available providers and a map 1508 illustrating the location of the providers. In addition, the interface 1500 provides filters 1506 that allow the bundled invasive and/or operative procedures in the list 1504 to be filtered and sorted in different manners. For example, the list 1504 can be sorted by price, location, etc. And, various provides can be selected and compared.

FIG. 17 shows an enlarged view of the map 1508, with a single provider 1602 highlighted. End users can select between different providers using the abbreviated list 1504.

Alternative embodiments are possible. For example, in another embodiment, the system can be used to accommodate bundled invasive and/or operative procedures associated with a particular provider or group of providers.

For example, a particular institution, such as a medical insurance provider and/or hospital, may desire to provide end users with information about the bundled invasive and/or operative procedures that the end user can select from the particular provider. In such a scenario, the system can be programmed to provide the end user with a branded experience associated with that particular provider. The end user accesses a site specifically-branded for the provider, and the end user is able to search for browse, and select one or more of the bundled invasive and/or operative procedures for that provider. This is accomplished by the system performing the methodology described herein and is transparent to the end user.

In such a “white-label” scenario, the provider can pay for the use of the system. The hosting of the data associated with the provider's bundled invasive and/or operative procedures can be handled and maintained by the system. In some examples, the provider can access the system to provide updates to the data associated with the bundled invasive and/or operative procedures listed within the system. In this example, the provider can be provided with a portal that allows the provider access to update data as well as receive information about the end users who have selected bundled invasive and/or operative procedures from the provider.

Referring now to FIG. 18, another example method 1800 for facilitating a bundled invasive and/or operative procedure is shown. In that example, various third party providers, such as a logistics provider, can be utilized to assemble the necessary resources for the bundle. Further, the logistics provider can be utilized to disassemble resources after the procedure is complete. Finally, payment for the various aspects of the bundle can be initiated in an automated and/or semi-automated manner through, for example, a financial institution. In this example, these functions are accomplished using a standard communications scheme, like EDI, that allows the system to communicate with third parties, such as the logistics provider, the financial institution, and/or the vehicle being used in transportation, which may be unmanned/robotic.

Referring now to step 1810 of the method 1800, a request for a bundled invasive and/or operative procedure is received, and the resources are selected that are needed for the selected bundled invasive and/or operative procedure. This can include determining on the back end which kind of participants are required, and determining on the back end which specific participants will participate in the procedure. This can also include identification of other resources needed for the bundle, such as one or more medical devices, like an implant.

Next, at operation 1820, one or more of the identified resources are converted into a common communication scheme format so that the system can communicate with a logistics provider to request those resources. In this example, the system converts the requests into EDI transactions that are communicated between the system and the logistics provider and/or its manned or unmanned transportation vehicles, i.e., drones. Such communications can include one or more of the following:
EDI purchase order transaction 850—a purchase order transaction set, which can be used to define a needed medical device (e.g., an implant) for a bundle;
EDI Motor Carrier Load Tender transaction 204—a transaction set used by shippers to tender an offer for a shipment to a full truckload motor carrier, which can be used to define shipment parameters for a needed medical device (e.g., an implant) for a bundle;
EDI motor carrier bill of lading transaction 211—a transaction set that provides a legal bill of lading for a shipment, such as the shipment of a needed medical device (e.g., an implant) for a bundle;
EDI transportation carrier shipment status message transaction 214—a transaction set used to specify the status of a shipment, such as the shipment of a needed medical device (e.g., an implant) for a bundle; and/or
EDI functional acknowledgment transaction 997—a transaction set that serves as a receipt acknowledging an EDI transaction.

These are examples of the possible communication scheme between the system and a logistics provider. The logistics provide can interact with the system through the EDI messages and provide the logistics necessary to stock, retrieve, and deliver necessary resources, such as implants, for the bundle. This delivery can occur to a specific place (e.g., the place of the procedure) and/or to a specific individual (e.g., the patient and/or the surgeon who will perform the procedure). Other configurations are possible.

Referring again to the method 1800, once the procedure services associated with the bundle are complete, control is passed to operation 1830, and the system receives and/or acknowledges completion. In one example, this can include the generation of an EDI message indicating completion of the procedure, such as an EDI general request, response, or confirmation transaction 814 and/or an EDI text message transaction 864.

In one example, the surgeon uses the EDI text message transaction 864 to indicate to the system once the procedure is complete. The message can, but need not always, indicate the status of the procedure and/or the work performed. Upon receipt of the EDI text message transaction 864, the system can perform various actions, such as communicating with the logistics provider for moving resources associated with the bundle, such as returning medical equipment and/or moving medical equipment to another site. Other configurations are possible. For example, in another embodiment, the system can be manually and/or automatically configured to determine when a procedure is complete and generate the necessary communications.

Finally, at operation 1840, the system is programmed to initiate payment for the services under the bundle. Such communications can include one or more of the following:
EDI invoice transaction 810—a transaction set that defines an electronic invoice for services rendered, such as the services of the caregiver site, surgeon, device (implant) manufacturer, and/or logistics provider;
EDI remittance advice transaction 820—a transaction set that defines information related to payment, such as defining an Automated Clearinghouse (ACH) banking transaction; and/or
EDI health care claim payment and remittance advice 835—a transaction set that is specified by HTPAA 5010 requirements for the electronic transmission of healthcare payment and benefit information.

Such communications can be directed to the provider(s) of the service(s) and/or to a financial institution. For example, the system can communicate with a financial institution to instruct payment for one or more of the providers (e.g., surgeon, medical device manufacturer, caregiver facility). Other configurations are possible.

Although various embodiments are described herein, those of ordinary skill in the art will understand that many modifications may be made thereto within the scope of the present disclosure. Accordingly, it is not intended that the scope of the disclosure in any way be limited by the examples provided.

What is claimed is:

1. A method implemented on an electronic computing device for presenting invasive and/or operative procedure bundles comprising:
receiving a request for an invasive and/or operative procedure bundle, the request identifying an end user and one or more services requested by the end user;
determining which kind of participants are required;
determining which specific participants will participate;
converting requests for the specific participants into electronic data interchange (EDI) transactions; and
communicating to the specific participants using the EDI transactions to define resource needs and deliver specifics.

2. The method of claim 1, further comprising:
determining that a medical device is required; and
sending a supply chain vendor an EDI purchase order transaction.

3. The method of claim 2, further comprising:
sending delivery instructions to an unmanned vehicle of the supply chain vendor.

4. The method of claim 1, further comprising:
determining that a medical device is required; and
sending a supply chain vendor an EDI Motor Carrier Load Tender transaction.

5. The method of claim 1, further comprising:
determining that a medical device is required; and
sending a supply chain vendor an EDI motor carrier bill of lading transaction.

6. The method of claim 1, further comprising:
determining that a medical device is required; and
sending a supply chain vendor an EDI transportation carrier shipment status message transaction.

7. The method of claim 1, further comprising:
determining that a medical device is required; and
sending a supply chain vendor an EDI functional acknowledgment transaction.

8. The method of claim 1, further comprising receiving an EDI general request, response, or confirmation transaction indicating procedure completion.

9. The method of claim 8, further comprising:
initiating of the EDI general request, response, or confirmation transaction by a surgeon.

10. The method of claim 1, further comprising receiving an EDI text message transaction indicating procedure completion.

11. The method of claim 10, further comprising:
allowing a surgeon to initiate the EDI text message transaction.

12. The method of claim 1, further comprising:
sending EDI invoice transaction to responsible payers.

13. The method of claim 1, further comprising:
sending EDI remittance advice transaction to communicate payment information to a service provider associated with the invasive and/or operative procedure bundle.

14. The method of claim 1, further comprising:
sending EDI payment instructions to a financial institution for paying a service provider associated with the invasive and/or operative procedure bundle.

15. A system for presenting invasive and/or operative procedure bundles, the system comprising:
at least one processor; and
memory encoding instructions that, when executed by the at least one processor, causes the at least one processor to:
receive a request for an invasive and/or operative procedure bundle, the request identifying an end user and one or more services requested by the end user;
determine which kind of participants are required;
determine which specific participants will participate;
convert requests for the specific participants into electronic data interchange (EDI) transactions; and
communicate to the specific participants using the EDI transactions to define resource needs and deliver specifics.

16. The system of claim 15, wherein the instructions are further encoded to cause the at least one processor to:
determine that a medical device is required; and
send a supply chain vendor an EDI purchase order transaction.

17. The system of claim 16, wherein the instructions are further encoded to cause the at least one processor to:
sending delivery instructions to an unmanned vehicle of the supply chain vendor.

18. The system of claim 15, wherein the instructions are further encoded to cause the at least one processor to:
determine that a medical device is required; and
send a supply chain vendor an EDI Motor Carrier Load Tender transaction.

19. The system of claim 15, wherein the instructions are further encoded to cause the at least one processor to:
determine that a medical device is required; and
send a supply chain vendor an EDI motor carrier bill of lading transaction.

20. The system of claim 15, wherein the instructions are further encoded to cause the at least one processor to:
determine that a medical device is required; and
send a supply chain vendor an EDI transportation carrier shipment status message transaction.
